(19) United States
(12) Patent Application Publication
(10) Pub. No.: US 2025/0223252 A1
(43) Pub. Date: Jul. 10, 2025

(54) METHOD FOR PRODUCING GLYCOLALDEHYDE DIALKYL ACETAL

(71) Applicant: TOYOTA JIDOSHA KABUSHIKI KAISHA, Toyota-shi (JP)

(72) Inventor: Wataru MURAMATSU, Susono-shi (JP)

(73) Assignee: TOYOTA JIDOSHA KABUSHIKI KAISHA, Toyota-shi (JP)

(21) Appl. No.: 18/947,709

(22) Filed: Nov. 14, 2024

(30) Foreign Application Priority Data
Jan. 10, 2024 (JP) 2024-001721

(51) Int. Cl. C07C 41/56

(52) U.S. Cl. CPC C07C 41/56; C07C 2521/18; C07C 2527/10; C07C 2527/24; C07C 2531/02

(57) ABSTRACT

The present disclosure provides means to produce glycolaldehyde dialkyl acetal in high yield. One aspect of the present disclosure relates to a method for producing glycolaldehyde dialkyl acetal including a glycolaldehyde formation step of converting paraformaldehyde to glycolaldehyde in an ether type solvent in a presence of an N-heterocyclic carbene catalyst, and an acetalization step of treating the glycolaldehyde with an alcohol solution of hydrogen chloride to obtain the glycolaldehyde dialkyl acetal. The N-heterocyclic carbene catalyst is a compound represented by a formula (I) or (II) [in the formulae, R¹, R², R³, R⁴, R⁵, R⁶, R⁷, and R⁸, and A⁻ have meanings described in description and the appended claims], and the glycolaldehyde formation step and the acetalization step are performed in a single container.

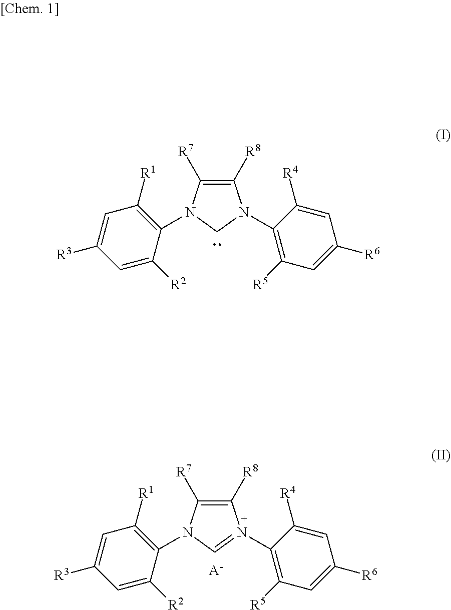

### CROSS REFERENCE TO RELATED APPLICATIONS

The present application claims priority from Japanese patent application JP 2024-001721 filed on Jan. 10, 2024, the entire content of which is hereby incorporated by reference into this application.

### BACKGROUND

### Technical Field

The present disclosure relates to a method for producing glycolaldehyde dialkyl acetal.

### Background Art

Glycolaldehyde is a dimerization product of formaldehyde. Glycolaldehyde can be used as a synthetic intermediate of useful compounds, such as amino acid, sugar, ethylene glycol, and ethanol. However, glycolaldehyde is highly reactive and easily dimerized. Therefore, it is difficult to isolate and storage glycolaldehyde.

Glycolaldehyde dialkyl acetal is obtained by acetalization of glycolaldehyde. Glycolaldehyde dialkyl acetal is more stable than glycolaldehyde. Therefore, glycolaldehyde dialkyl acetal is valuable as a raw material for producing useful compounds.

For example, JP H06-211723 A discloses a catalytical preparation of condensation products of formaldehyde in which formaldehyde or a formaldehyde-forming compound is caused to undergo reaction using a catalyst which has been produced, in the presence of an auxiliary base, from a triazolium salt or a tetrazolium salt.

JP H01-272543 A discloses, as hydroformylation of aqueous formaldehyde using a rhodium-tricyclophosphine catalyst system, a process for reacting formaldehyde, carbon monoxide, and hydrogen in the presence of a rhodium complex catalyst under hydroformylation conditions to form glycol aldehyde.

### SUMMARY

Since glycolaldehyde is highly reactive and easily dimerized, more stable glycolaldehyde dialkyl acetal is expected as a raw material for producing useful compounds. However, a method for producing glycolaldehyde dialkyl acetal in high yield has not been known.

Accordingly, the present disclosure provides means to produce glycolaldehyde dialkyl acetal in high yield.

The present inventors examined various means to solve the above-described problem. The present inventors found that desired glycolaldehyde dialkyl acetal can be obtained in high yield by converting paraformaldehyde to glycolaldehyde using a specific N-heterocyclic carbene catalyst and then performing a reaction for acetalizing the glycolaldehyde in a single container. The present inventors completed the present disclosure based on the finding.

That is, the present disclosure includes the following aspects and embodiments.
(Embodiment 1) A method for producing glycolaldehyde dialkyl acetal, comprising:
   converting paraformaldehyde to glycolaldehyde in an ether type solvent in a presence of an N-heterocyclic carbene catalyst; and
   treating the glycolaldehyde with an alcohol solution of hydrogen chloride to obtain the glycolaldehyde dialkyl acetal,
   wherein the N-heterocyclic carbene catalyst is a compound represented by a formula (I) or (II):

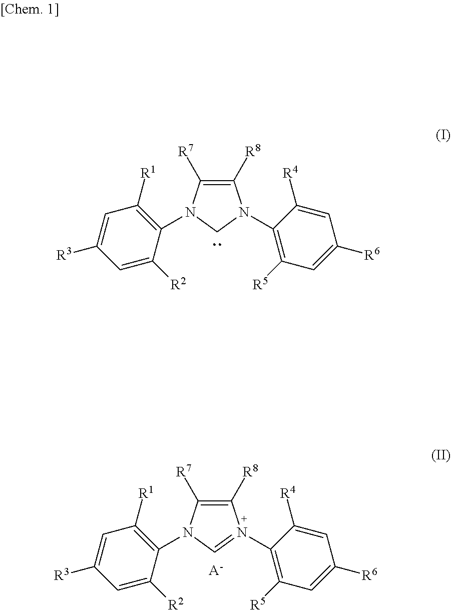

[in the formulae,
   R¹ and R⁴ are mutually independently unsubstituted C₃-C₆ alkyl or C₁-C₆ alkyl substituted by C₆-C₁₈ aryl,
   R² and R⁵ are mutually independently unsubstituted C₃-C₆ alkyl or C₁-C₆ alkyl substituted by C₆-C₁₈ aryl,
   R³ and R⁶ are mutually independently H, unsubstituted C₁-C₆ alkoxy, unsubstituted C₃-C₆ alkyl, or C₁-C₆ alkyl substituted by C₆-C₁₈ aryl,
   R⁷ and R⁸ are both H or form C₆-C₁₈ aryl together with a carbon atom bonded thereto, and
   A⁻ is halogen anion or carbon dioxide radical anion], and
   wherein the converting and the treating are performed in a single container.
(Embodiment 2) The method according to Embodiment 1,
   wherein the ether type solvent is selected from the group consisting of cyclopentyl methyl ether (CPME), tert-butyl methyl ether (TBME), diethyl ether, 1,4-dioxane, tetrahydrofuran (THF), 2-methyltetrahydrofuran (2-MeTHF), and anisole.
(Embodiment 3) The method according to Embodiment 1 or 2,
   wherein the method is performed in the presence of the N-heterocyclic carbene catalyst in which
   R¹ and R⁴ are mutually independently unsubstituted C₃-C₆ alkyl,
   R² and R⁵ are mutually independently unsubstituted C₃-C₆ alkyl,
   R³ and R⁶, and R⁷ and R⁸ are all H, and
   A⁻ is halogen anion or carbon dioxide radical anion.
(Embodiment 4) The method according to any of Embodiments 1 to 3
   wherein the method is performed in the presence of the N-heterocyclic carbene catalyst in which
   R¹ and R⁴, and R² and R⁵ are all isopropyl,
   R³ and R⁶, and R⁷ and R⁸ are all H, and
   A⁻ is chlorine anion or carbon dioxide radical anion.
(Embodiment 5) The method according to any of Embodiments 1 to 4,
   wherein the ether type solvent is cyclopentyl methyl ether (CPME), tert-butyl methyl ether (TBME), diethyl ether, or 1,4-dioxane, and
   wherein the N-heterocyclic carbene catalyst is 1,3-bis(2,6-diisopropylphenyl) imidazol-2-ylidene, 1,3-bis(2,6-diisopropylphenyl) imidazolium chloride, or 1,3-bis(2,6-diisopropylphenyl) imidazolium-2-carboxylate.

The present disclosure can provide the means to produce glycolaldehyde dialkyl acetal in high yield.

### DETAILED DESCRIPTION

The following describes embodiments of the present disclosure in detail.

One aspect of the present disclosure relates to a method for producing glycolaldehyde dialkyl acetal. The method of this aspect includes a glycolaldehyde formation step and an acetalization step. The following describes respective steps in detail.

### [1: Glycolaldehyde Formation Step]

This step includes converting paraformaldehyde to glycolaldehyde in an ether type solvent in the presence of an N-heterocyclic carbene catalyst.

In this step, the N-heterocyclic carbene catalyst is a compound represented by a formula (I) or (II):

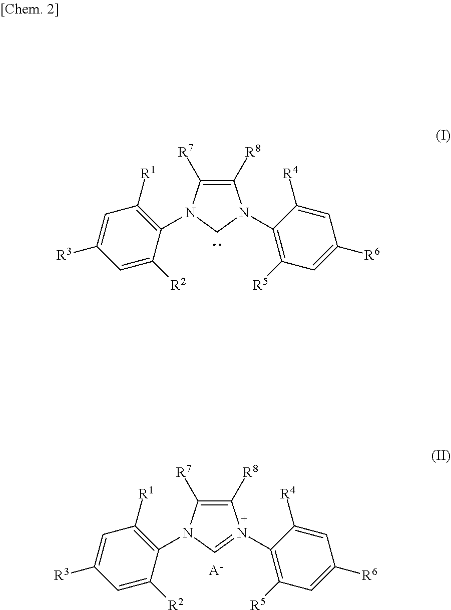

In the formulae (I) and (II),
R¹ and R⁴ are mutually independently unsubstituted C₃-C₆ alkyl or C₁-C₆ alkyl substituted by C₆-C₁₈ aryl,
R² and R⁵ are mutually independently unsubstituted C₃-C₆ alkyl or C₁-C₆ alkyl substituted by C₆-C₁₈ aryl,
R³ and R⁶ are mutually independently H, unsubstituted C₁-C₆ alkoxy, unsubstituted C₃-C₆ alkyl, or C₁-C₆ alkyl substituted by C₆-C₁₈ aryl,
R⁷ and R⁸ are both H or form C₆-C₁₈ aryl together with a carbon atom bonded thereto, and
A⁻ is halogen anion or carbon dioxide radical anion.

In the formulae (I) and (II), R¹ and R⁴ are mutually independently unsubstituted C₃-C₆ alkyl, R² and R⁵ are mutually independently unsubstituted C₃-C₆ alkyl, R³ and R⁶, and R⁷ and R⁸ are all H, and A⁻ is halogen anion or carbon dioxide radical anion in some embodiments. R¹ and R⁴, and R² and R⁵ are mutually independently isopropyl, tert-butyl, or sec-butyl, R³ and R⁶, and R⁷ and R⁸ are all H, and A⁻ is chlorine anion, bromine anion, iodine anion, or carbon dioxide radical anion in some embodiments. R¹ and R⁴, and R² and R⁵ are all isopropyl, R³ and R⁶, and R⁷ and R⁸ are all H, and A is chlorine anion or carbon dioxide radical anion in some embodiments.

When R¹ and R⁴, and R² and R⁵ are the above-described groups in the formulae (I) and (II), their three-dimensional bulky structures allow suppressing an undesired side reaction, such as dimerization. Accordingly, glycolaldehyde can be obtained in high yield and/or with high selectivity.

The N-heterocyclic carbene catalyst used in this step is 1,3-bis(2,6-diisopropylphenyl) imidazol-2-ylidene (that is, in the formula (I), R¹ and R⁴, and R² and R⁵ are all isopropyl, and R³ and R⁶, and R⁷ and R⁸ are all H), 1,3-bis(2,6-diisopropylphenyl) imidazolium chloride (that is, in the formula (II), R¹ and R⁴, and R² and R⁵ are all isopropyl, R³ and R⁶, and R⁷ and R⁸ are all H, and A is chlorine anion), or 1,3-bis(2,6-diisopropylphenyl) imidazolium-2-carboxylate (that is, in the formula (II), R¹ and R⁴, and R² and R⁵ are all isopropyl, R³ and R⁶, and R⁷ and R⁸ are all H, and A is carbon dioxide radical anion) in some embodiments. By performing this step using the above-described N-heterocyclic carbene catalyst, glycolaldehyde can be obtained in especially high yield and/or with high selectivity.

When the N-heterocyclic carbene catalyst used in this step is a compound represented by the formula (II), the compound is used with a base in some embodiments. The base is selected from the group consisting of triethylamine, diisopropylethylamine, 1,8-diazabicyclo[5.4.0]undec-7-ene (DBU), 1,5-diazabicyclo[4.3.0]-5-nonene (DBN), 7-methyl-1,5,7-triazabicyclo[4.4.0]dec-5-ene (MTBD), lithium diisopropylamide (LDA), and phosphazene base in some embodiments, and is DBU, DBN, MTBD, LDA, or phosphazene base in some embodiments. Alternatively, the base may be a base in immobilized form supported on a carrier, such as a resin. Examples of the base in immobilized form includes a base in immobilized form in which the above-described base is bonded to a carrier, and an ion-exchange resin. By performing this step using the N-heterocyclic carbene catalyst represented by the formula (II) together with the above-described base, glycolaldehyde can be obtained in high yield.

The N-heterocyclic carbene catalyst used in this step is in a range of from 0.01 to 5 mol % relative to the number of moles of paraformaldehyde as a raw material in some embodiments, in a range of from 0.05 to 2 mol % in some embodiments, and in a range of from 0.1 to 1 mol % in some embodiments. By performing this step using the N-heterocyclic carbene catalyst at an amount in the above-described range, glycolaldehyde can be obtained in high yield.

In this step, the ether type solvent is selected from the group consisting of cyclopentyl methyl ether (CPME), tert-butyl methyl ether (TBME), diethyl ether, 1,4-dioxane, tetrahydrofuran (THF), 2-methyltetrahydrofuran (2-MeTHF), and anisole in some embodiments, and is cyclopentyl methyl ether (CPME), tert-butyl methyl ether (TBME), diethyl ether, or 1,4-dioxane in some embodiments. By performing this step using the above-described ether type solvent, glycolaldehyde can be obtained in high yield. The above-described ether type solvent is low-price, and known to be industrially safe. Therefore, by performing this step using the above-described ether type solvent, glycolaldehyde can be obtained at low-price and/or to be industrially safe.

A reaction temperature and a reaction time in this step may be appropriately set based on the boiling point of the ether type solvent to be used. The reaction temperature is a temperature equal to or more than room temperature in some embodiments, 25° C. or more in some embodiments, and 30° C. or more in some embodiments. The reaction temperature is a temperature exceeding the boiling point of the ether type solvent to be used in some embodiments, 100° C. or less in some embodiments, and 90° C. or less in some embodiments. The reaction time is five minutes or more in some embodiments, 10 minutes or more in some embodiments, and 30 minutes or more in some embodiments. The reaction time is 200 minutes or less in some embodiments, 100 minutes or less in some embodiments, and 60 minutes or less in some embodiments. By performing this step under the above-described reactive conditions, glycolaldehyde can be obtained in high yield.

### [2: Acctalization Step]

This step includes treating glycolaldehyde with an alcohol solution of hydrogen chloride to obtain glycolaldehyde dialkyl acetal.

In this step, an alcohol solvent of the alcohol solution of hydrogen chloride is selected from the group consisting of methanol, ethanol, benzyl alcohol, and phenol in some embodiments, and is methanol in some embodiments. By performing this step using the alcohol solution of hydrogen chloride containing the above-described alcohol solvent, glycolaldehyde dialkyl acetal can be obtained in high yield.

The concentration of the alcohol solution of hydrogen chloride used in this step is in a range of from 0.01 to 1 M relative to the number of moles of paraformaldehyde as a raw material in some embodiments, and is in a range of from 0.05 to 0.1 M in some embodiments. By performing this step using the alcohol solution of hydrogen chloride at the concentration in the above-described range, glycolaldehyde dialkyl acetal can be obtained in high yield.

In the method of this aspect, the glycolaldehyde formation step and the acetalization step are performed in a single container. The glycolaldehyde formation step and the acetalization step are continuously performed in the single container in some embodiments. For example, after performing the glycolaldehyde formation step, the acetalization step is directly performed in the same reaction container without purifying a reaction mixture containing glycolaldehyde in some embodiments. Glycolaldehyde that is a product in the glycolaldehyde formation step is highly reactive and easily dimerized. Therefore, when the glycolaldehyde formation step and the acetalization step are performed in different containers (that is, sequentially performed), an undesired side reaction, such as dimerization of glycolaldehyde, possibly progresses before performing the acetalization step. Accordingly, by performing the glycolaldehyde formation step and the acetalization step in the single container, the undesired side reaction can be substantially suppressed, and glycolaldehyde dialkyl acetal can be obtained in high yield.

As described above in detail, by the method of this aspect, glycolaldehyde dialkyl acetal can be produced in high yield. Glycolaldehyde dialkyl acetal obtained by the method of this aspect is expected as a raw material for producing useful compounds, such as amino acid, sugar, ethylene glycol, and ethanol. Accordingly, the method of this aspect can provide the raw material of these useful compounds.

### EXAMPLES

The following further specifically describes the present disclosure using examples. However, the technical scope of the present disclosure is not limited to these examples.

### Experiment 1

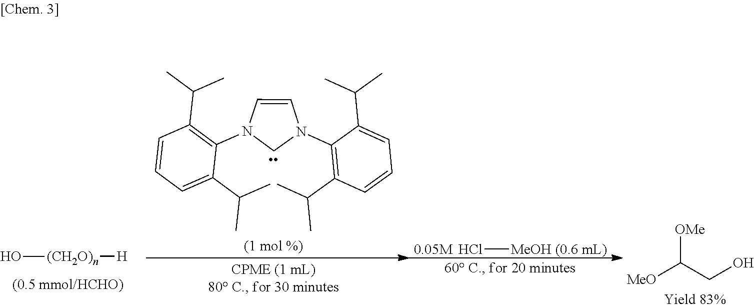

In a reaction container, paraformaldehyde (0.5 mmol/HCHO) was reacted in a cyclopentyl methyl ether (CPME) solvent (1 mL) at 80° C. for 30 minutes in the presence of a 1,3-bis(2,6-diisopropylphenyl) imidazol-2-ylidene catalyst (1 mol %), thereby converting paraformaldehyde to glycolaldehyde (glycolaldehyde formation step). Without purifying a reaction mixture containing glycolaldehyde, treating was directly performed in the same reaction container with a methanol solution (0.6 mL) of 0.05 M of hydrogen chloride at 60° C. for 20 minutes (acetalization step). Glycolaldehyde dialkyl acetal contained in the obtained crude reaction product was analyzed with 1H NMR and GC. By the reaction, glycolaldehyde dialkyl acetal was obtained in the yield of 83%.

### Experiment 2: Solvent Optimization

Glycolaldehyde dialkyl acetal was synthesized by a procedure similar to Experiment 1 excluding that the solvent was changed to those indicated below in the procedure of Experiment 1. The used solvents and the yield (%) of glycolaldehyde dialkyl acetal are indicated in Table 1.

TABLE 1

| | Yield |
|---|---|
| Solvent | (%) |
| tert-butyl methyl ether (TBME) | 69 |
| diethyl ether | 53 |
| 1,4-dioxane | 58 |
| tetrahydrofuran (THF) | 61 |
| 2-methyltetrahydrofuran (2-MeTHF) | 40 |
| toluene | 32 |
| diisopropyl ethyl ether | 14 |
| anisole | <1 |
| dimethyl ether | <1 |
| ethyl acetate | 20 |
| trifluoroethanol | <1 |
| dichloromethane | 19 |
| N,N-diethylformamide | <1 |
| N-methylpyrrolidone | <1 |
| methanol | <1 |
| tert-butanol | 7 |
| water | <1 |
| N,N-dimethylformamide | <1 |
| dimethylacetamide | <1 |
| acetonitrile | 5 |

As illustrated in Table 1, by performing the glycolaldehyde formation step using the ether type solvents, glycolaldehyde dialkyl acetal was obtained in high yield.

### Experiment 3: Catalyst Optimization (1)

Glycolaldehyde dialkyl acetal was synthesized by a procedure similar to Experiment 1 excluding that the catalyst was changed in the procedure of Experiment 1. When an N-heterocyclic carbene catalyst in which a side chain group bonded to nitrogen atom of imidazole ring of the catalyst used in Experiment 1 is an alkyl group, an N-heterocyclic carbene catalyst in which the imidazole ring is imidazolidine ring, and an N-heterocyclic carbene catalyst in which a side chain group of phenyl bonded to nitrogen atom of the imidazole ring is a methyl group or a diphenylmethyl group were used, the reaction did almost not progress in any on the cases (yield of less than 4%). When a triphenyltriazole ylidene catalyst (JP H06-211723 A) was used, the reaction did almost not progress as well (yield of less than 1%).

### Experiment 4: Reaction Scale Up and Catalytic Amount Reduction

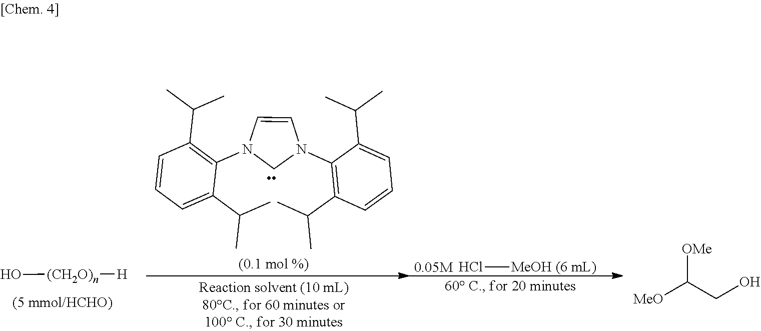

Glycolaldehyde dialkyl acetal was synthesized by a procedure similar to Experiment 1 excluding that the reactive condition was changed as described in the scheme in the procedure of Experiment 1. The used solvents, the reactive conditions, and the yield (%) of glycolaldehyde dialkyl acetal are indicated in Table 2. In the table, a reactive condition (a) is a condition that the glycolaldehyde formation step is performed at 80° C. for 60 minutes, and a reactive condition (b) is a condition that the glycolaldehyde formation step is performed at 100° C. for 30 minutes.

TABLE 2

| | Yield (%) | |
|---|---|---|
| Solvent | Reactive Condition (a) | Reactive Condition (b) |
| 1,4-dioxane | 74 | 72 |
| CPME | 72 | 71 |
| TBME | 47 | 73 |
| THF | 46 | 49 |

As illustrated in Table 2, even when the catalytic amount is reduced to 1/10 compared with Experiment 1, by raising the reaction temperature or increasing the reaction time, glycolaldehyde dialkyl acetal was able to be obtained in high yield.

### Experiment 5: Catalyst Optimization (2)

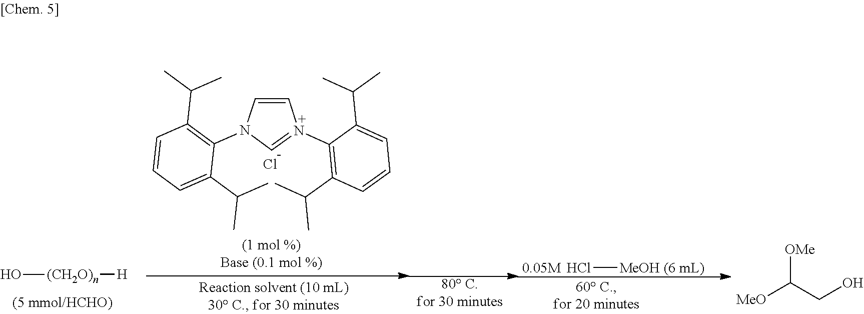

Glycolaldehyde dialkyl acetal was synthesized by a procedure similar to Experiment 1 excluding that the catalyst was changed to a combination of the above-described imidazole salt and a base, and the reactive condition was changed as described in the scheme in the procedure of Experiment 4. The used bases and solvents, and the yield (%) of glycolaldehyde dialkyl acetal are indicated in Table 3. In the table, DBU is 1,8-diazabicyclo[5.4.0]undec-7-ene, DBN is 1,5-diazabicyclo[4.3.0]-5-nonene, MTBD is 7-methyl-1,5,7-triazabicyclo[4.4.0]dec-5-ene, and LDA is lithium diisopropylamide.

TABLE 3

| | | Yield |
|---|---|---|
| Base | Solvent | (%) |
| DBU | 1,4-dioxane | 72 |
| DBN | 1,4-dioxane | 63 |
| MTBD | 1,4-dioxane | 71 |
| LDA | 1,4-dioxane | 70 |
| DBU | CPME | 70 |
| DBN | CPME | 62 |
| MTBD | CPME | 60 |
| LDA | CPME | 68 |

As illustrated in Table 3, by performing the glycolaldehyde formation step using the combination of the imidazole salt and the base as the catalyst, glycolaldehyde dialkyl acetal was able to be obtained in high yield.

### Experiment 6: Catalyst Optimization (3)

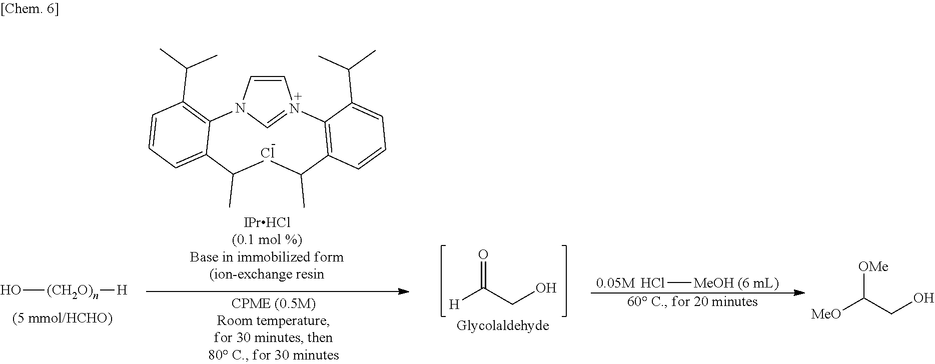

Glycolaldehyde dialkyl acetal was synthesized by a procedure similar to Experiment 1 excluding that the catalyst was changed to a combination of the above-described imidazole salt and a base in immobilized form (ion-exchange resin), and the reactive condition was changed as described in the scheme in the procedure of Experiment 4. The used bases and the yield (%) of glycolaldehyde dialkyl acetal are indicated in Table 4.

TABLE 4

| | Amount of Use | Yield |
|---|---|---|
| Base | (weight %) | (%) |
| DBU supported on PS resin | 2.2 | 63 |
| 2-tert-butylimino-2-diethylamino-1,3- | 2.2 | 71 |
| dimethylperhydro-1,3,2-diazaphosphorine | | |
| supported on PS resin | | |
| trimethylamine supported on PS resin | 3.3 | 50 |
| (Muromac XMA-413B-OH) | | |

As illustrated in Table 4, even when the glycolaldehyde formation step was performed using the combination of the imidazole salt and the base in immobilized form as the catalyst, glycolaldehyde dialkyl acetal was able to be obtained in high yield.

### Experiment 7: Catalyst Optimization (4)

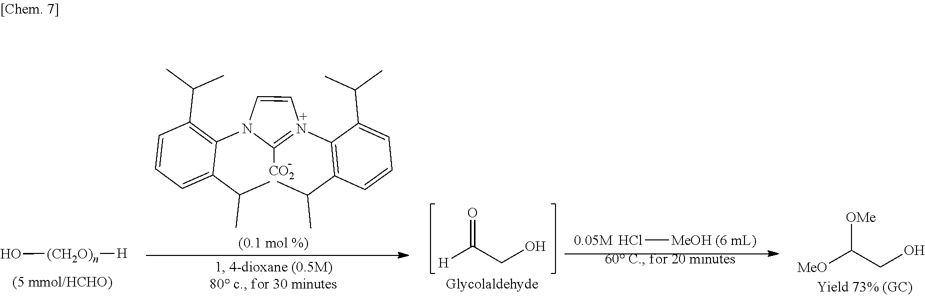

Glycolaldehyde dialkyl acetal was synthesized by a procedure similar to Experiment 1 excluding that the catalyst was changed to 1,3-bis(2,6-diisopropylphenyl) imidazolium-2-carboxylate, and the reactive condition was changed as described in the scheme in the procedure of Experiment 4. Glycolaldehyde dialkyl acetal contained in the obtained crude reaction product was analyzed with GC. By the reaction, glycolaldehyde dialkyl acetal was obtained in the yield of 73%.

The present disclosure is not limited to the above-described examples, and includes various modifications. The examples described above are described in detail to facilitate understanding of the present disclosure and are not necessarily limited to those having all the described configurations. It is also possible to add, delete, and/or replace a part of the configuration of each example with another configuration.

What is claimed is:

1. A method for producing glycolaldehyde dialkyl acetal, comprising:
converting paraformaldehyde to glycolaldehyde in an ether type solvent in a presence of an N-heterocyclic carbene catalyst; and
treating the glycolaldehyde with an alcohol solution of hydrogen chloride to obtain the glycolaldehyde dialkyl acetal,
wherein the N-heterocyclic carbene catalyst is a compound represented by a formula (I) or (II): 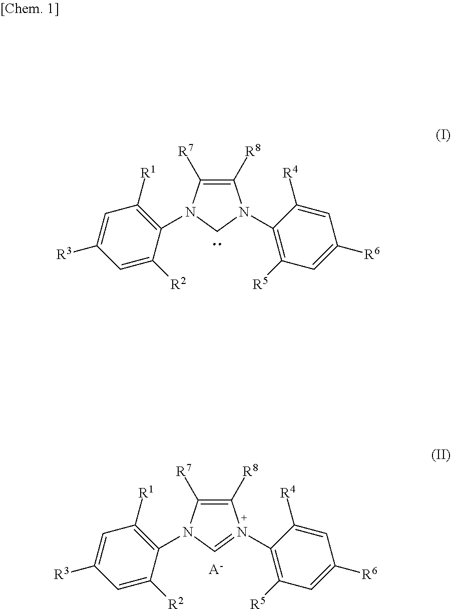
[in the formulae,
R¹ and R⁴ are mutually independently unsubstituted C₃-C₆ alkyl or C₁-C₆ alkyl substituted by C₆-C₁₈ aryl,
R² and R⁵ are mutually independently unsubstituted C₃-C₆ alkyl or C₁-C₆ alkyl substituted by C₆-C₁₈ aryl,
R³ and R⁶ are mutually independently H, unsubstituted C₁-C₆ alkoxy, unsubstituted C₃-C₆ alkyl, or C₁-C₆ alkyl substituted by C₆-C₁₈ aryl,
R⁷ and R⁸ are both H or form C₆-C₁₈ aryl together with a carbon atom bonded thereto, and
A⁻ is halogen anion or carbon dioxide radical anion], and
wherein the converting and the treating are performed in a single container.

2. The method according to claim 1,
wherein the ether type solvent is selected from the group consisting of cyclopentyl methyl ether (CPME), tert-butyl methyl ether (TBME), diethyl ether, 1,4-dioxane, tetrahydrofuran (THF), 2-methyltetrahydrofuran (2-MeTHF), and anisole.

3. The method according to claim 1,
wherein the method is performed in the presence of the N-heterocyclic carbene catalyst in which
R¹ and R⁴ are mutually independently unsubstituted C₃-C₆ alkyl,
R² and R⁵ are mutually independently unsubstituted C₃-C₆ alkyl,
R³ and R⁶, and R⁷ and R⁸ are all H, and
A⁻ is halogen anion or carbon dioxide radical anion.

4. The method according to claim 1,
wherein the method is performed in the presence of the N-heterocyclic carbene catalyst in which
R¹ and R⁴, and R² and R⁵ are all isopropyl,
R³ and R⁶, and R⁷ and R⁸ are all H, and
A⁻ is chlorine anion or carbon dioxide radical anion.

5. The method according to claim 1,
wherein the ether type solvent is cyclopentyl methyl ether (CPME), tert-butyl methyl ether (TBME), diethyl ether, or 1,4-dioxane, and
wherein the N-heterocyclic carbene catalyst is 1,3-bis(2,6-diisopropylphenyl) imidazol-2-ylidene, 1,3-bis(2,6-diisopropylphenyl) imidazolium chloride, or 1,3-bis(2,6-diisopropylphenyl) imidazolium-2-carboxylate.
